(19) United States
(12) Patent Application Publication
(10) Pub. No.: US 2016/0030579 A1
(43) Pub. Date: Feb. 4, 2016

(54) Stabilization of Essential Oils Within a Hydrocolloid Adhesive

(71) Applicant: AVERY DENNISON CORPORATION, Glendale, CA (US)

(72) Inventor: Neal Carty, Mentor, OH (US)

(73) Assignee: AVERY DENNISON CORPORATION, Glendale, CA (US)

(21) Appl. No.: 14/776,629

(22) PCT Filed: Mar. 13, 2014
(86) PCT No.: PCT/US14/25221

§ 371 Date: Sep. 14, 2015

Related U.S. Application Data
(60) Provisional application No. 61/781,069, filed on Mar. 14, 2013.

(51) Int. Cl. A61K 47/32; A61K 47/38; A61K 47/44; A61L 31/04; A61L 15/24; A61L 27/16; A61L 29/04; A61K 31/192; A61K 47/10

(52) U.S. Cl. CPC A61K 47/32; A61K 31/192; A61K 47/38; A61K 47/44; A61K 47/10; A61L 15/24; A61L 27/16; A61L 29/041; A61L 31/048

(57) ABSTRACT

Various adhesive compositions are described which comprise one or more essential oils. The adhesive compositions may optionally comprise one or more active agents such as pharmaceutical agents. Also described are related methods of improving the stability of essential oil(s) in adhesive compositions by incorporating polyvinylpyrrolidone. Also described are related methods of using the compositions and articles incorporating such compositions.

### CROSS REFERENCES TO RELATED APPLICATIONS

The present application claims the benefit of U.S. Provisional Application No. 61/781,069 filed Mar. 14, 2013, which is incorporated herein by reference in its entirety.

### FIELD

The present subject matter relates to adhesive compositions. In certain versions, the subject matter relates to improving stability of adhesive compositions that include one or more essential oils. The subject matter is particularly directed to medical adhesives containing one or more essential oils and optionally in combination with pharmaceutical active agents.

### BACKGROUND

Adhesive compositions are known which introduce one or more active agents for release when placed in contact with human skin tissue. Vehicles or solvents are used to solubilize or suspend one or more active agents therein and to introduce the actives into the adhesive. Absorbents may be used in the compositions to absorb, retain, or transmit moisture or aqueous agents, which may be desirable in some compositions. Although satisfactory in certain respects, the combination of these components tends to inhibit and degrade the adhesive properties of the compositions.

It is also known to incorporate one or more essential oils in adhesive compositions. Essential oils have been utilized in a wide array of personal care products. Essential oils can be used to impart certain smells, tastes, or other properties to a personal care composition. However, in certain applications, the essential oil can degrade or otherwise lose one or more of its desirable properties.

Accordingly, a need exists for essential oil-containing adhesive compositions that exhibit improved and prolonged stability and storage characteristics, absorb or retain moisture, and at the same time exhibit improved adhesive properties for the composition as a whole, and which can be readily tailored and utilized in a variety of different applications.

### SUMMARY

The difficulties and drawbacks associated with previously known compositions are addressed by the compositions, articles, and related methods of the present subject matter as follows.

In one aspect, the present subject matter provides a method of enhancing the stability of essential oils in an adhesive composition. The adhesive composition comprises an adhesive component, at least one essential oil, and an absorbent. The method comprises providing polyvinylpyrrolidone and incorporating the polyvinylpyrrolidone in the adhesive composition, whereby the stability of the essential oils is enhanced.

In another aspect, the present subject matter provides an adhesive composition comprising an adhesive component, at least one essential oil, an absorbent, and polyvinylpyrrolidone.

In yet another aspect, the present subject matter provides an article for adhesive attachment to a surface of interest. The article comprises a substrate defining at least one face, and a region of an adhesive composition disposed on at least a portion of the face of the substrate. The adhesive composition includes (i) an adhesive component, (ii) at least one essential oil, (iii) an absorbent, and (iv) polyvinylpyrrolidone.

As will be realized, the subject matter is capable of other and different embodiments and its several details are capable of modifications in various respects, all without departing from the subject matter. Accordingly, the description is to be regarded as illustrative and not restrictive.

### DETAILED DESCRIPTION OF THE EMBODIMENTS

The present subject matter relates to a discovery that incorporation of polyvinylpyrrolidone (PVP) in a hydrocolloid adhesive containing absorbent and one or more essential oils, promotes maintaining or stabilizing of the essential oil in the adhesive. Without the PVP, the essential oil tends to evaporate from the adhesive composition.

More specifically, the present subject matter is directed to enhancing stability properties of an essential oil-containing adhesive composition comprising one or more absorbents by incorporation of PVP in the adhesive composition. The subject matter is generally directed to pressure sensitive adhesives or hot melt adhesives but can encompass other types of adhesives. Although the present subject matter is primarily directed to adhesive compositions that contain one or more essential oils, it will be understood that the subject matter also encompasses adhesives which include combinations of essential oils with one or more actives. The compositions of the present subject matter may also comprise one or more polyhydric alcohols. These and other aspects of the present subject matter are described in greater detail herein.

The present subject matter provides a wide array of adhesive compositions which include one or more essential oils and optionally in combination with one or more active agent(s). Table 1 set forth below lists representative adhesive compositions in accordance with the present subject matter. All percentages expressed herein are percentages by weight, unless noted otherwise.

TABLE 1
Adhesive Compositions

Weight Percentage

| Component | Generally | Particularly |
|---|---|---|
| Adhesive Component | 20%-90% | 40%-60% |
| Absorbent(s) | 1%-50% | 5%-20% |
| Essential Oil(s) | 0.1%-20% | 0.5%-5% |
| Polyvinylpyrrolidone | 0.1%-30% | 1%-10% |
| Polyhydric Alcohol(s) | 0.1%-30% | 5%-25% |
| (Optional) | | |

### Adhesive(s)

A wide array of adhesive components can be used in the adhesive compositions according to the present subject matter. Generally, the adhesive or adhesive component is a hot melt adhesive. In certain aspects, the present subject matter compositions comprise a hot melt pressure sensitive adhesive that is able to be processed at a temperature below 75° C. In some aspects the hot melt adhesive will soften at temperatures between about 60° C. and about 70° C. for low temperature processing so as to avoid the breakdown or degradation of active agents.

The adhesive matrix may be based on for example polyisobutylene, butyl rubber, polyacrylates, polyurethanes, silicone gum, natural gum rubber, SBR rubber or polyvinyl ether. Thermoplastic elastomers such as styrene-isoprene-styrene block copolymers and styrene-ethylene/propylene-styrene block copolymers may be used, and these may require optional tackifiers and plasticizers. Blends or mixtures of elastomers may be more easily employed.

Particularly suitable as bases for the pressure sensitive adhesives of the present subject matter are rubbers such as linear or radial A-B-A block copolymers or mixtures of these A-B-A block copolymers with simple A-B block copolymers. These block copolymers can be based on styrene-butadiene, styrene-isoprene, and hydrogenated styrene-diene copolymers such as styrene ethylene-butylene.

Suitable styrene-diene copolymers for the practice of the present subject matter are exemplified by a blend of linear styrene/isoprene/styrene triblock copolymer and linear styrene/isoprene diblock copolymer. Such a material is available from Shell Chemical as Kraton D-1161 and has a bound styrene content of about 15% and a diblock content of 17%. A second example is a blend of linear styrene/isoprene/styrene triblock copolymer and linear styrene/isoprene diblock copolymer available from Shell Chemical as Kraton D-1117 and which has a bound styrene content of about 17% and a diblock content of 33%.

An example of a suitable hydrogenated styrene-diene copolymer is a thermoplastic elastomer comprising a blend of clear linear triblock and diblock copolymer based on styrene and ethylene/butylene, with a bound styrene of 14% mass. Such a material is commercially available from Shell Chemical Company as Kraton G-1657. Another example is Kraton G-1652 from Shell Chemical Company, which is a thermoplastic elastomer comprised of a clear linear triblock copolymer based on styrene and ethylene-butylene, S-E/B-S, with a bound styrene content of about 30% by weight. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer may be suitable. Such a material is available from Shell Chemical Company as Kraton Research Product RP6919. This material has a styrene content of 18%, an isoprene content of 36% and an ethylene-butylene content of 46% by weight. Also, a low styrene synthetic copolymer of butadiene and styrene, commonly called SBR rubber, can be used as a solid rubber.

Also particularly suitable are acrylic pressure sensitive adhesives, exemplified by an acrylic hot melt adhesive manufactured by Schenectedy Chemicals and having the designation Durotac 401. Another example is an acrylic solvent adhesive from Avery Chemicals called Polytex 7600.

### Absorbent(s)

Similarly, a wide array of absorbents can be utilized in the adhesive compositions according to the present subject matter. Generally, the absorbent includes one or more hydrophilic polymers that are soluble or insoluble but swellable in water, as the moisture-absorbing component. Suitable insoluble swellable polymers include cross-linked sodium carboxymethyl cellulose, crystalline sodium carboxymethyl cellulose, cross-linked dextran and starch-acrylonitrile graft copolymer. The swellable polymer may also be a so-called “super absorbent” material such as starch sodium polyacrylate. Other hydratable polymers such as gluten and polymers of methyl vinyl ether and maleic acid and derivatives thereof may also be included. Suitable water soluble polymers include sodium carboxymethyl cellulose, pectin, gelatin, guar gum, locust bean gum, collagen, tragacanth gum, karaya gum starches, gum arabic, alginic acid and various sodium and/or calcium salts thereof. Other synthetic absorbents such as polyvinyl alcohol, polyvinyl acetate, polyvinyl pyrollidone, polyacrylic acid, polyhydroxyalkyl acrylates, polyacrylamides, high molecular weight polyethylene glycols and polypropylene glycols may be useful.

The super absorbent polymer (SAP), if used in the adhesive compositions, comprises a water-swellable, hydrogel-forming absorbent polymer capable of absorbing large quantities of liquids such as water, body fluids (e.g., urine, blood), and the like. Additionally, the SAP is capable of retaining such absorbed fluids under moderate pressures. Typically the SAP absorbs many times its own weight in water, preferably at least 50 times, more preferably at least 100 times, most preferably at least 150 times its weight in water. Additionally, the SAP exhibits good saline fluid absorption under load and high saline fluid absorption capacity. Typically the SAP absorbs at least 10 times, preferably at least 30 times, more preferably at least 50 times its weight in saline fluid. Even though the SAP is capable of absorbing many times its own weight in water and/or saline, it does not dissolve in these fluids.

The ability of the SAP to absorb water and/or saline fluid is related to the degree of crosslinking present in the SAP. Increasing the degree of crosslinking increases the SAP's total fluid holding capacity under load. The degree of crosslinking is preferably optimized to obtain a composition in which the rate and amount of absorbency are optimized. Useful SAPs are at least 10%, more preferably from about 10% to about 50%, and most preferably from about 20% to 40% crosslinked. Examples of suitable SAPs include crosslinked and polymerized α,β-beta ethylenically unsaturated mono- and dicarboxylic acids and acid anhydride monomers including, e.g., acrylic acid, methacrylic acid, crotonic acid, maleic acid/anhydride, itaconic acid, fumaric acid, and combinations thereof.

Super absorbent polymers useful in the present subject matter include, e.g., crosslinked acrylate polymers, crosslinked products of vinyl alcohol-acrylate copolymers, crosslinked products of polyvinyl alcohols grafted with maleic anhydride, cross-linked products of acrylate-methacrylate copolymers, crosslinked saponification products of methyl acrylate-vinyl acetate copolymers, crosslinked products of starch acrylate graft copolymers, crosslinked saponification products of starch acrylonitrile graft copolymers, crosslinked products of carboxymethyl cellulose polymers and crosslinked products of isobutylene-maleic anhydride copolymers, and combinations thereof.

The super absorbent polymer(s) is typically in the form of particles and preferably are spherical and have an average particle size of from about 1 micrometer (μm) to about 400 (μm).

Preferably the particles have an average particle size of from about 20 μm to about 200 μm, and more preferably from 20 μm to 150 μm. In one embodiment, the particle size of the particles is less than 150 μm, or less than 100 μm. Useful commercially available super absorbent particles include, e.g., sodium polyacrylate super absorbent particles available under the AQUA KEEP series of trade designations including, e.g., particles having an average particle size of from about 20 μm to about 30 μm available under the trade designation AQUA KEEP 1 OSH-NF, particles having an average particle size of from 200 μm to 300 μm available under the trade designation AQUA KEEP 10SH-P, particles having an average particle size of from 320 μm to 370 μm available under the trade designation AQUA KEEP SA60S, particles having an average particle size of from 350 μm to 390 μm available under the trade designations AQUA KEEP SA60SX, SA55SX π and SA 60SL II, and particles having an average particle size of from 250 μm to 350 μm available under the trade designation AQUA KEEP SA6ON TYPE II from Sumitomo Seika Chemicals Co., Ltd. (Japan). Also available super absorbent materials are Luquasorb 1010 and Luquasorb 1030 from BASF, Ludwigshafen, Germany.

Thus in summary, the absorbent(s) utilized in the adhesive compositions of the present subject matter is typically one or more agents selected from (i) insoluble swellable polymers, (ii) hydratable polymers, (iii) water soluble polymers, (iv) synthetic absorbents, (v) super absorbent polymers, and/or (vi) combinations of any one or more of (i)-(v).

For certain embodiments, it is useful to utilize one or more types or grades of carboxymethyl cellulose (CMC) in the present subject matter compositions and methods. CMC is a cellulose ether comprised of repeating cellobiose units. These are composed of two anhydroglucose units (beta-glucopyranose residues). A parameter used in referring to grades of CMC is the degree of polymerization. This is the number of anhydroglucose units which are joined through 1, 4 glucosidic linkages. Each anhydroglucose unit contains three hydroxyl groups. By substituting carboxymethyl groups for some of the hydrogens of the hydroxyl groups, sodium carboxymethyl cellulose is obtained. The average number of hydroxyl groups substituted per anhydroglucose unit is known as the “degree of substitution.” If all three hydroxyls are replaced, the maximum theoretical degree of substitution is 3.0 (impossible to practice).

Another parameter used in reference to CMC is average chain length or degree of polymerization. Average chain length (or the degree of polymerization) and the previously noted degree of substitution determine molecular weight of the CMC polymer.

For many embodiments, the CMC utilized in the present subject matter has a degree of substitution of from about 0.2 to about 1.5, and in other embodiments from about 0.7 to about 1.2. In particular embodiments, the degree of substitution of the CMC is from about 0.65 to about 0.90. The molecular weight of CMC is typically within a range of from about 17,000 to about 700,000. The present subject matter includes CMC grades having molecular weights less than 17,000 and greater than 700,000.

In certain versions of the present subject matter, a particularly useful absorbent is sodium carboxymethyl cellulose commercially available from various sources such as from Ashland Chemical under the designation AQUASORB A500. It is also contemplated that instead of, or in addition to, carboxymethyl cellulose; hydroxypropyl cellulose, hydroxypropylmethyl cellulose, and variants thereof can be used in the present subject matter.

It will be understood that although the present subject matter relates to absorbent adhesives, it is contemplated that the present subject matter could also be applicable to adhesives that are free of absorbents. Thus, in certain versions, the subject matter includes stabilizing and/or promoting retention of essential oil(s) in an adhesive composition that does not contain an absorbent as described herein.

### Essential Oil(s)

The adhesive compositions of the present subject matter include at least one essential oil. Useful essential oils non-exclusively include cineol (eucalyptol), thymol, menthol, methyl salicylate, wintergreen oil, carvacrol, camphor, anethole, carvone, eugenol, isoeugenol, limonene, osimen, n-decyl alcohol, citronel, α-salpineol, methyl acetate, citronellyl acetate, methyl eugenol, linalaol, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, star anise, or combinations thereof. Additional nonlimiting examples of essential oils include almond oil, caraway oil, cardamom oil, celery oil, chamomile oil, coriander oil, corn oil, cottonseed oil, cumin oil, dill oil, fennel oil, garlic oil, geranium oil, ginger oil, grapefruit oil, lime oil, linseed oil, mint oil, parsley oil, pepper oil, rose oil, sesame oil, soybean oil, turmeric oil, or any of the natural or synthetic active ingredients in essential oils such as ethyl salicylate, propyl salicylate, safrole, and D-limonene. In certain versions of the present subject matter, a particular essential oil useful in an adhesive composition is wintergreen oil. In one embodiment, the essential oil component(s) may be present in the overall composition in an amount of from about 0.10% to about 20%. In another embodiment, the essential oil component(s) may be present in the overall composition in an amount of from about 0.50% to about 5%.

### Optional Active Agent(s)

Furthermore, a wide array of active agents can optionally be used in the compositions of the present subject matter. Generally, any active, active agent, or combination of actives and/or active agents which are biologically active and which can be incorporated within the adhesive composition in a stable manner or form, can be utilized. It will be understood that the active(s) are optional. In certain versions of the present subject matter, the active agent is soluble in the vehicle and particularly in polyhydric alcohol(s) when such are utilized as vehicles in the compositions. In certain versions of the present subject matter, the active agent forms a complex with the polyvinylpyrrolidone or other agents, described in greater detail herein. The complex typically results from hydrogen bonding between the active(s) and the inhibitor(s).

In certain aspects, the active(s) can be for example the pain relievers or analgesics fentanyl, butorphanol, morphine, buprenorphine, naloxone, codeine; local anaesthetics such as lidocaine, anti-acne drugs like retinoic acid; anti-angina drugs like nitroglycerin, isosorbide dinitrate, nifedipine, nicardipine; antiarrhythmics like timolol; antibacterials like amikacin, cephalosporins, macrolides, tetracyclines, quinolones, nitrofurantoin; anti-convulsives like carbamazepine, phenobarbital, nitrazepam; antidepressants like tricyclics, bupropion, sertraline, pergolide, fluoxetine; anti-rheumatics like diclofenac, ibuprofen, piroxicam, ketoprofen, thiocolchicosicle, methotrexate; sex hormones like progesterone, testosterone, estradiol, levonorgestrel; anti-fungals like clotrimazole, ketoconazole, miconazole; anti-hypertensives like sotalol, alprenolol, captopril, enalapril, felodipine, nicardipine, reserpine; anti-hypothyroid drugs like thyroxine; anti-malarials like artemesine, cinchonidine, primaquine; anti-migraine drugs like ergotamine, sumatriptan, rizatriptan; anti-nausea drugs like domperidone, chlorpromazine, methoclopramide, scopolamine, tetrahydrocannabinoids; skin lighteners like hydroquinone, hydroquinine; dopamine receptor antagonists like pergolide, bromocriptine; muscle relaxants like thiocolchicoside, diazepam; sclerosing agents like ethanolamine, sodium ricinoleate; vitamins like A, B, C, E and precursors or various agents like oxybutynin, finasteride, erythropoetine. Combinations of one or more of these actives are also contemplated including combinations of these agents with still other ingredients.

### Polyvinylpyrrolidone

Polyvinylpyrrolidone (PVP) is a white, hygroscopic polymer with a weak characteristic odor. PVP is usually in powder form, although it can be in solution, and comprises the monomer N-vinylpyrrolidone as the base unit. By selecting suitable polymerization conditions, a wide range of molecular weights can be obtained, extending from low values of a few thousand daltons to approximately 2.2 million daltons, i.e. 2,200 kDa.

PVP can be either a homopolymer or copolymer, typically synthesized by free-radical polymerization in water or alcohols with a suitable initiator of vinylpyrrolidone (also referred to as N-vinylpyrrolidone, N-vinyl-2-pyrrolidone and N-vinyl-2-pyrrolidinone) as a monomeric unit. PVP polymers include soluble and insoluble homopolymeric PVPs, and copolymers such as vinylpyrrolidone/vinyl acetate and vinylpyrrolidone/dimethylamino-ethylmethacrylate. Substantially cross-linked homopolymers of PVP are insoluble and are generally known in the pharmaceutical industry under the designations polyvinylpolypyrrolidone, crospovidone and PVP. The copolymer vinylpyrrolidone-vinyl acetate is generally known in the pharmaceutical industry under the designations Copolyvidon(e), Copolyvidonum or VP-VAc.

In certain aspects of the present subject matter, the PVP is soluble. The term “soluble” when used with reference to PVP means that the polymer is soluble in water and generally is not substantially cross-linked, and has a weight average molecular weight of less than about 2,200,000. In contrast to most polymers, soluble PVP is readily soluble in water and also in a large number of organic solvents, such as alcohols, amines, acids, chlorinated hydrocarbons, amides and lactams. Soluble PVP polymers have been identified in the pharmaceutical industry under a variety of names, the most commonly used include Povidone, Polyvidon(e), Polyvidonum, Polyvidonum, poly(N-vinyl-2-pyrrolidinone, poly(N-vinylbutyrolactam), poly(1-vinyl-2-pyrrolidone), poly[1-(2-oxo-1-pyrrolidinyl)ethylene]. PVP homopolymer is generally insoluble in the common esters, ethers, hydrocarbons and ketones. When dry, soluble PVP homopolymer is a light flaky powder, which absorbs up to 40% of its weight in water.

The amount and type of PVP required in the embodiments typically depends on the particular application of the adhesive composition, as well as the type of adhesives and agents contained therein. Typically, the PVP is present in an amount from about 0.10% to about 30% by weight of the weight of the total adhesive composition. The soluble PVP for certain versions of the present subject matter has a weight average molecular weight of less than about 2,200 kilodaltons (kDa), more particularly less than about 100 kDa, and most particularly less than 54 kDa. In certain versions, it is useful to employ PVP having a weight average molecular weight from about 2,000 to 2,200,000 (i.e. 2 kDa to 2,200 kDa), more particularly from 5,000 to 100,000 (i.e. 5 kDa to 100 kDa), and most particularly 7,000 to 54,000 (i.e. 7 kDa to 54 kDa). In certain versions of the present subject matter it is useful to employ PVP having certain characteristics and/or properties. For example, in certain embodiments, the PVP has a weight average molecular weight (Mw) of from about 9 to about 850 kilodaltons (kDa), and a number average molecular weight (Mn) of from about 2 to about 200 kDa. In certain aspects, the PVP has a glass transition temperature of from about 110° C. to about 180° C. And, in certain embodiments, the PVP has a K-value of from about 15 to about 82. It is also contemplated to utilize PVP exhibiting all of these characteristics.

In certain versions of the present subject matter, it is advantageous to utilize one or more commercially available grades of PVP, such as those under the trade name LUVITEC®, LUVICROSS®, KOLLINDON®, and COLLACRAL®VAL from BASF Corporation; and PLASDONE, POLYPLASDONE and COPOLYMER 958 by ISP Technologies, Wayne, N.J.

BASF offers a wide range of suitable soluble vinylpyrrolidone homopolymers with different molecular weights (K-values) under the name LUVITEC® K. The products are available as a powder or as aqueous solutions. Characteristic parameters of all LUVITEC® K grades are listed in Table 2.

TABLE 2
Representative Grades of PVP

| | K-value: | | pH-value | Residual NVP | Brookfield-RVT |
|---|---|---|---|---|---|
| | Mw in | | (10% | content in | Viscosity in |
| | kDa | Solids content in % | solution) | ppm | mPa · s at 23° C. |
| LUVITEC ® K 17 | 15-19 | 95.0-100.0 | 3.0-7.0 | ≦100 | 80-180 |
| powder | | | | | (40/2/100) |
| LUVITEC ® K 30 | 27-33 | 95.0-100.0 | 3.0-7.0 | ≦100 | 80-140 |
| powder | | | | | (30/1/50) |
| LUVITEC ® K 80 | 74-82 | 95.0-100.0 | 5.0-8.0 | ≦100 | 2500-7000 |
| powder | | | | | (20/6/100) |
| LUVITEC ® K 85 | 84-88 | 95.0-100.0 | 5.0-9.0 | ≦100 | 5000-20000 |
| powder | | | | | (20/6/50) |
| LUVITEC ® K 90 | 88-92 | 95.0-100.0 | 5.0-9.0 | ≦100 | 10000-25000 |
| powder | | | | | (20/7/100) |
| LUVITEC ® K 90 HM | 92-96 | 95.0-100.0 | 5.0-9.0 | ≦100 | 15000-30000 |
| powder | | | | | (20/7/100) |
| LUVITEC ® K 30 | 27-33 | 29.0-31.0 | 4.0-8.0 | ≦100 | 80-140 |
| solution approx. 30% | | | | | (30/1/50) |
| LUVITEC ® K 60 | 52-62 | 34.0-36.0 | 7.0-9.0 | ≦300 | 2000-20000 |
| solution approx. 35% | | | | | (35/6/50) |
| LUVITEC ® K 85 CQ | 83-88 | 19.0-21.0 | 7.0-9.0 | ≦100 | 5000-15000 |
| solution approx. 20% | | | | | (20/6/50) |
| LUVITEC ® K 90 CQ | 90-98 | 9.5-10.5 | 7.0-9.0 | ≦50 | 300-1500 |
| solution approx. 10% | | | | | (10/4/100) |
| LUVITEC ® K 90 | 90-98 | 19.0-21.0 | 7.0-9.0 | ≦100 | 10000-40000 |
| solution approx. 20% | | | | | (20/7/100) |
| LUVITEC ® K 115 CQ | 114-130 | 10.5-11.5 | 7.0-9.0 | ≦50 | 2000-5000 |
| solution approx. 10% | | | | | (11/6/100) |

Table 3 set forth below presents typical properties of various grades of PVP commercially available under the LUVITEC® trade name.

TABLE 3
Typical Properties of PVP

| LUVITEC ® | K 17 | K 30 | K 60 | K 80 | K 85 | K 90 | K 90 HM | K 115 |
|---|---|---|---|---|---|---|---|---|
| Molecular weight | | | | | | | | |
| (GPC): | | | | | | | | |
| Mw in kDa | 9 | 50 | 450 | 850 | 1100 | 1400 | 1800 | 2200 |
| Mn in kDa | 2 | 14 | 140 | 200 | 250 | 325 | 375 | 400 |
| Ash content in % | | | | ≦0.02 | | | | |
| Rel. Viscosity | 1.09 | 1.25 | 1.93 | 3.09 | 3.74 | 5.09 | 5.69 | 12.1 |
| (1% in water, 23° C. | (5%: | | | | | | | (0.1%: |
| capillary viscometer) | 1.53) | | | | | | | 1.33) |
| Glass transition | 110 | 175 | 175 | 180 | 180 | 180 | 180 | 180 |
| temperature in ° C. | | | | | | | | |
| (DSC) | | | | | | | | |
| Particle size in μm | | | | | | | | |
| (Sympatec-Helos | | | | | | | | |
| Rodos) | | | | | | | | |
| X₁₀% | 15 | 25 | Only | 60 | 90 | 90 | 90 | Only |
| X₅₀% | 25 | 75 | available | 160 | 180 | 180 | 180 | available |
| X₉₀% | 100 | 130 | as | 320 | 350 | 350 | 350 | as |
| | | | solution | | | | | solution |

| | |
|---|---|
| Color (10% solution, | Brighter than BY5/B5/R6 |
| according to Europ. | |
| Pharmacopoeia) | |
| Moisture absorption | 20 (50% rel. humidity, 23° C.) |
| at saturation in % | 40 (75% rel. humidity, 23° C.) |

In certain versions of the present subject matter, the soluble PVP homopolymer used in the present subject matter is a low molecular weight PVP (for example having a molecular weight less than about 60 kDa) that can be used either alone or in combination with other soluble PVP homopolymers or with other crystallization inhibitors. In one aspect the soluble PVP homopolymer used has a molecular weight from about 0.1 kDa to about 54 kDa, more particularly from about 8 kDa to about 10 kDa).

### Optional Polyhydric Alcohol(s)

Polyhydric alcohols can optionally be utilized in the present subject matter compositions. Although not wishing to be bound to any particular theory, it is believed that incorporating a polyhydric alcohol such as glycerol, provides a humectant or hydrating effect upon skin. In certain versions of the present subject matter, use of one or more polyhydric alcohols also serves to plasticize or dissolve the PVP so that the PVP can be processed at temperatures below the typical glass transition temperature(s) of the PVP. Suitable examples of polyhydric alcohols include dihydric alcohols, such as ethylene glycol, propylene glycol, 1,3- and 1,4-butanediols, 1,6-hexanediol, methylene oxidepentyl glycol, diethylene glycol, bis(hydroxymethyl)cyclohexane, bis(hydroxyethyl)benzene, hydrogenated bisphenol A, hydrogenated bisphenol F, polytetramethylene glycols, polyester dials and silanol-terminated polysiloxanes; trihydric alcohols, such as glycerol, trirnethylol propane, trimethylol ethane, 1,2,3-butane triol, 1,2,6-hexane triol and polyester triols; and polyhydric alcohols having 4 to 8 or more hydroxyl groups, such as pentaerythritol, diglycerol, α-methylglucoside, sorbitol, xylitol, mannitol, volernitol, erythritol, threitol, glucose, fructose, sucrose, and the like.

It is contemplated that other agents can potentially be used in combination with the polyhydric alcohol(s). For example, other optional agents include monovalent and multivalent alcohols with up to 24 carbon atoms, such as 1,2-propanediol, 1,3-propanediol, 1,2-ethanediol, glycerol or lauryl alcohol; free carboxylic acids with up to 24 carbon atoms, such as lauric acid; fatty acid esters with up to 24 carbon atoms in the fatty acid component and up to 20 carbon atoms in the monovalent or multivalent alcohol component, such as isopropyl myristate, glycerol monopalmitate, dodecanoyl acetate; terpenes, amides, urea and mixtures of these penetration enhancers. In certain versions of the present subject matter, the compositions may include poly-glycols such as polyethylene glycols and/or polypropylene glycols. However, it will be appreciated that the present subject matter includes the use of other vehicles, carriers, and/or solvents instead of, or in addition to those mentioned herein.

### Additional Aspects

In certain embodiments, the adhesive composition is a hydrocolloid adhesive. A base hydrocolloid adhesive formulation generally comprises a hot melt adhesive blended with an absorbent such as sodium carboxymethylcellulose (CMC), gelatin, pectin, alginate, polyacrylate superabsorbent, or the like. Other hydrocarbon resins, such as polyisobutylene, can also be included to adjust adhesive properties. Any of these formulations can be selected for the base adhesive in a drug-delivery application in accordance with the present subject matter.

For effective drug delivery it is advantageous to incorporate a polyhydric alcohol which acts primarily as a vehicle into which the drug is actually dissolved, but may also serve a secondary function as a skin penetration enhancer. Propylene glycol is an example of a polyhydric alcohol which serves both purposes. Other examples of polyhydric alcohol vehicles include glycerol and polyethylene glycols, typically with molecular weights between 200 and 1,000 Da, or any mixtures thereof.

As explained herein, it is advantageous to include polyvinylpyrrolidone (PVP), which generally includes soluble PVP homopolymers of low molecular weight in the adhesive compositions containing essential oils. Incorporation of PVP has been found to stabilize and/or promote retention of the essential oils within the composition.

Although the adhesive compositions of the present subject matter generally stabilize and/or retain essential oil(s) contained in the compositions, the compositions in many instances can also deliver or transmit essential oil(s) to an adjacent substrate such as biological skin. For example, in certain versions of the present subject matter, the adhesives deliver at least 5% of the essential oil(s) in the adhesive within a time period of 8 hours.

### Methods

The present subject matter also provides various methods. In one version, a method of stabilizing or promoting retention of essential oils in an adhesive composition is provided. The adhesive composition comprises an adhesive that includes one or more absorbents, and essential oils. The method further includes incorporating PVP in the adhesive composition. The adhesive, absorbent(s), essential oil(s), and PVP are as described herein. The various components can be incorporated with one another, blended, and/or otherwise combined in techniques or operations known in the art.

### Medical Articles

The adhesive compositions described herein can be used in association with a wide array of medical articles. Nonlimiting examples of such articles include wound dressings, surgical dressings, medical tapes, athletic tapes, surgical tapes, sensors, electrodes, ostomy appliances or related components such as sealing rings, catheters, connector fittings, catheter hubs, catheter adapters, fluid delivery tubes, electrical wires and cables, negative pressure wound therapy (NPWT) components, surgical drains, wound draining components, IV site dressings, prostheses, stoma pouches, buccal patches, transdermal patches, pain relieving patches, wrinkle reduction patches, dentures, hairpieces, bandages, diapers, medical padding for example liposuction padding, hygiene pads, corn and callous pads, toe cushioning pads, and pads for protecting and cushioning tube sites such as tracheotomy tubes. The medical articles include one or more regions or surfaces to which the adhesive compositions of the present subject matter are applied. Forming a layer, coating, or other region of adhesive on an article enables the article to be adhered to a wide range of surfaces, including skin. It will be understood that the present subject matter is not limited to any of these articles. Instead, the subject matter includes the use of the adhesive compositions with other articles besides those noted herein. The medical articles may also include one or more layers covering the adhesive layer or coating such as a release liner.

Many other benefits will no doubt become apparent from future application and development of this technology.

All patents, applications, standards, and articles noted herein are hereby incorporated by reference in their entirety.

As described hereinabove, the present subject matter solves many problems associated with previous strategies, systems or devices. However, it will be appreciated that various changes in the details, materials and arrangements of components and operations, which have been herein described and illustrated in order to explain the nature of the subject matter, may be made by those skilled in the art without departing from the principle and scope of the subject matter, as expressed in the appended claims.

What is claimed is:

1. A method of enhancing the stability of essential oils in an adhesive composition, wherein the adhesive composition comprises an adhesive component, at least one essential oil, and an absorbent, the method comprising:
providing polyvinylpyrrolidone;
incorporating the polyvinylpyrrolidone in the adhesive composition, whereby the stability of the essential oils is enhanced.

2. The method of claim 1, wherein the absorbent is selected from the group consisting of insoluble swellable polymers, hydratable polymers, water soluble polymers, synthetic absorbents, super absorbent polymers, and combinations thereof

3. The method of claim 2, wherein the absorbent is carboxymethyl cellulose.

4. The method of claim 3, wherein the carboxymethyl cellulose has a degree of substitution within a range from 0.2 to 1.5.

5. The method of any one of claims 3-4, wherein the carboxymethyl cellulose has a molecular weight in a range from 17,000 to 700,000.

6. The method of any one of claims 1-5 wherein the adhesive composition further comprises an active agent.

7. The method of claim 6, wherein the active agent is selected from the group consisting of analgesics, local anesthetics, anti-acne agents, anti-angina agents, antiarrhythmics, antibacterial, anti-convulsives, antidepressants, anti-rheumatics, sex hormones, anti-fungals, anti-hypertensives, anti-hypothyroid agents, anti-malarials, anti-migraine agents, anti-nausea agents, skin lighteners, dopamine receptor antagonists, muscle relaxants, sclerosing agents, vitamins and combinations thereof.

8. The method of claim 7, wherein the active agent includes an anti-rheumatic.

9. The method of claim 8, wherein the anti-rheumatic is ibuprofen.

10. The method of any one of claims 1-9 wherein the at least one essential oil is selected from the group consisting of cineol (eucalyptol), thymol, menthol, methyl salicylate, wintergreen oil, carvacrol, camphor, anethole, carvone, eugenol, isoeugenol, limonene, osimen, n-decyl alcohol, citronel, α-salpineol, methyl acetate, citronellyl acetate, methyl eugenol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, star anise, almond oil, caraway oil, cardamom oil, celery oil, chamomile oil, coriander oil, corn oil, cottonseed oil, cumin oil, dill oil, fennel oil, garlic oil, geranium oil, ginger oil, grapefruit oil, lime oil, linseed oil, mint oil, parsley oil, pepper oil, rose oil, sesame oil, soybean oil, turmeric oil, or combinations thereof.

11. The method of any one of claims 1-9 wherein the essential oil is wintergreen oil.

12. The method of any one of claims 1-11, wherein the adhesive composition further comprises a polyhydric alcohol.

13. The method of claim 12, wherein the polyhydric alcohol is selected from the group consisting of propylene glycol, glycerol, polyethylene glycol, and combinations thereof.

14. The method of claim 13, wherein the polyhydric alcohol includes propylene glycol.

15. The method of claim 13 wherein the polyhydric alcohol includes glycerol.

16. The method of any one of claims 1-15, wherein the adhesive component is a hot melt adhesive.

17. The method of any one of claims 1-16 wherein the adhesive compositions comprises 20% to 90% of the adhesive component, from 0.1% to 20% of the essential oil(s), and from 1% to 50% of an absorbent.

18. The method of any one of claims 1-17 wherein the polyvinylpyrrolidone is incorporated in the adhesive composition in an amount of 0.1% to 30%.

19. The method of any one of claims 1-18, wherein the incorporating includes bringing the adhesive composition to a temperature between about 60° C. and about 70° C. to soften the adhesive.

20. An adhesive composition comprising:
an adhesive component;
at least one essential oil;
an absorbent; and
polyvinylpyrrolidone.

21. The adhesive composition of claim 20 further comprising a polyhydric alcohol.

22. The adhesive composition of any one of claims 20-21, wherein the absorbent is selected from the group consisting of insoluble swellable polymers, hydratable polymers, water soluble polymers, synthetic absorbents, super absorbent polymers, and combinations thereof.

23. The adhesive composition of claim 22, wherein the absorbent is carboxymethyl cellulose.

24. The adhesive composition of claim 23, wherein the carboxymethyl cellulose has a degree of substitution within a range from 0.2 to 1.5.

25. The adhesive composition of any one of claims 23-24, wherein the carboxymethyl cellulose has a molecular weight in a range from 17,000 to 700,000.

26. The adhesive composition of any one of claims 20-25 further comprising:
at least one active agent.

27. The adhesive composition of claim 26, wherein the active agent is selected from the group consisting of analgesics, local anesthetics, anti-acne agents, anti-angina agents, antiarrhythmics, antibacterial, anti-convulsives, antidepressants, anti-rheumatics, sex hormones, anti-fungals, anti-hypertensives, anti-hypothyroid agents, anti-malarials, anti-migraine agents, anti-nausea agents, skin lighteners, dopamine receptor antagonists, muscle relaxants, sclerosing agents, vitamins and combinations thereof.

28. The adhesive composition of claim 27, wherein the active agent includes an anti-rheumatic.

29. The adhesive composition of claim 28, wherein the anti-rheumatic is ibuprofen.

30. The adhesive composition of any one of claims 21-29, wherein the polyhydric alcohol is selected from the group consisting of propylene glycol, glycerol, polyethylene glycol, and combinations thereof.

31. The adhesive composition of any one of claims 20-30 wherein the at least one essential oil is selected from the group consisting of cineol (eucalyptol), thymol, menthol, methyl salicylate, wintergreen oil, carvacrol, camphor, anethole, caryone, eugenol, isoeugenol, limonene, osimen, n-decyl alcohol, citronel, α-salpineol, methyl acetate, citronellyl acetate, methyl eugenol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, star anise, almond oil, caraway oil, cardamom oil, celery oil, chamomile oil, coriander oil, corn oil, cottonseed oil, cumin oil, dill oil, fennel oil, garlic oil, geranium oil, ginger oil, grapefruit oil, lime oil, linseed oil, mint oil, parsley oil, pepper oil, rose oil, sesame oil, soybean oil, turmeric oil, or combinations thereof.

32. The adhesive composition of any one of claims 20-30 wherein the essential oil is wintergreen oil.

33. The adhesive composition of any one of claims 20-32 wherein the composition comprises 20% to 90% of the adhesive component, from 0.1% to 20% of the essential oil(s), from 1% to 50% of an absorbent, and from 0.1% to 30% of the polyvinylpyrrolidone.

34. An article for adhesive attachment to a surface of interest, the article comprising:
a substrate defining at least one face; and
a region of an adhesive composition disposed on at least a portion of the face of the substrate, the adhesive composition including (i) an adhesive component, (ii) at least one essential oil, (iii) an absorbent, and (iv) polyvinylpyrrolidone.

35. The article of claim 34 wherein the article is a medical article.

36. The article of claim 35 wherein the medical article is selected from the group consisting of wound dressings, surgical dressings, medical tapes, athletic tapes, surgical tapes, sensors, electrodes, ostomy appliances, sealing rings, catheters, connector fittings, catheter hubs, catheter adapters, fluid delivery tubes, electrical wires and cables, negative pressure wound therapy (NPWT) components, surgical drains, wound draining components, IV site dressings, prostheses, stoma pouches, buccal patches, transdermal patches, wrinkle reduction patches, dentures, hairpieces, bandages, diapers, medical padding, liposuction padding, hygiene pads, corn and callous pads, toe cushioning pads, and pads for protecting and cushioning tube sites such as tracheotomy tubes.

37. The article of any one of claims 34-36 wherein the adhesive composition further includes a polyhydric alcohol.

38. The article of claim 37, wherein the polyhydric alcohol is selected from the group consisting of propylene glycol, glycerol, polyethylene glycol, and combinations thereof.

39. The article of claim 38 wherein the polyhydric alcohol is propylene glycol.

40. The article of any one of claims 34-39 wherein the adhesive composition further includes at least one active agent.

41. The article of any one of claims 34-40, wherein the absorbent is selected from the group consisting of insoluble swellable polymers, hydratable polymers, water soluble polymers, synthetic absorbents, super absorbent polymers, and combinations thereof.

42. The article of claim 41, wherein the absorbent is carboxymethyl cellulose.

43. The article of claim 42, wherein the carboxymethyl cellulose has a degree of substitution within a range from 0.2 to 1.5.

44. The article of any one of claims 42-43, wherein the carboxymethyl cellulose has a molecular weight in a range from 17,000 to 700,000.

45. The article of any one of claims 40-44, wherein the active agent is selected from the group consisting of analgesics, local anesthetics, anti-acne agents, anti-angina agents, antiarrhythmics, antibacterial, anti-convulsives, antidepressants, anti-rheumatics, sex hormones, anti-fungals, anti-hypertensives, anti-hypothyroid agents, anti-malarials, anti-migraine agents, anti-nausea agents, skin lighteners, dopamine receptor antagonists, muscle relaxants, sclerosing agents, vitamins and combinations thereof.

46. The article of claim 45, wherein the active agent includes an anti-rheumatic.

47. The article of claim 46, wherein the anti-rheumatic is ibuprofen.

48. The article of any one of claims 34-47 wherein the at least one essential oil is selected from the group consisting of cineol (eucalyptol), thymol, menthol, methyl salicylate, wintergreen oil, carvacrol, camphor, anethole, carvone, eugenol, isoeugenol, limonene, osimen, n-decyl alcohol, citronel, a-salpineol, methyl acetate, citronellyl acetate, methyl eugenol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, star anise, almond oil, caraway oil, cardamom oil, celery oil, chamomile oil, coriander oil, corn oil, cottonseed oil, cumin oil, dill oil, fennel oil, garlic oil, geranium oil, ginger oil, grapefruit oil, lime oil, linseed oil, mint oil, parsley oil, pepper oil, rose oil, sesame oil, soybean oil, turmeric oil, or combinations thereof.

49. The article of any one of claims 34-47 wherein the essential oil is wintergreen oil.

50. The article of any one of claims 34-49 wherein the composition comprises 20% to 90% of the adhesive component, from 0.1% to 20% of the essential oil(s), from 1% to 50% of an absorbent, and from 0.1% to 30% of the polyvinylpyrrolidone.
